(19) United States
(12) Patent Application Publication
(10) Pub. No.: US 2016/0017398 A1
(43) Pub. Date: Jan. 21, 2016

(54) PROCESSES FOR BIOCONVERSION OF CARBON BEARING MATERIALS

(71) Applicant: CIRIS ENERGY, INC., Centennial, CO (US)

(72) Inventors: Jay M. Short, Centennial, CO (US); Robert Bartek, Centennial, CO (US)

(73) Assignee: Ciris Energy, Inc., Centennial, CO (US)

(21) Appl. No.: 14/773,832

(22) PCT Filed: Mar. 13, 2014
(86) PCT No.: PCT/US2014/026586

§ 371 Date: Sep. 9, 2015

Related U.S. Application Data
(60) Provisional application No. 61/792,798, filed on Mar. 15, 2013.

(51) Int. Cl. C12P 39/00; C12P 5/02

(52) U.S. Cl. CPC C12P 39/00; C12P 5/023

(57) ABSTRACT

A process involving a microorganism consortium for converting at least one component in a carbon-bearing material to a different product comprising at least one hydrocarbon. In the process, a microorganism consortium is contacted with a composition that causes an increase or decrease of a relative population of at least one species of microorganism in said microorganism consortium, to enhance a yield or selectivity or alter a rate of said process. The composition may be selected from a composition that affects an intracellular pathway of said at least one species of microorganism, a composition that affects an intercellular signaling pathway that involves said at least one species of microorganism and at least one antisense RNA. Also, the microorganism consortium can be exposed to signals such as sound waves or electromagnetic signals or a condition of the environment of the microorganism consortium can be altered.

### BACKGROUND

1. Field

The present disclosure relates to bioconversion of carbon bearing materials of geologic formations. In particular, the present disclosure is directed to introducing a substance into the carbon bearing material to enhance one or more aspects of a bioconversion process.

2. Description of Related Technology

Increasing world energy demand is creating unprecedented challenges for recovering energy resources, and mitigating the environmental impact of using those resources. Historically, subterraneous formations such as old oil fields and coal mines are abandoned once easily recoverable materials are extracted. These abandoned reservoirs, however, still contain significant amounts of carbon bearing materials. The Powder River Basin in northeastern Wyoming, for example, is still estimated to contain approximately 1,300 billion short tons of coal. Just 1% of the Basin's remaining coal converted to natural gas could supply the current annual natural gas needs of the United States (i.e., about 23 trillion cubic feet) for the next four years. Several other abandoned coal and oil reservoirs of this magnitude are present in the United States.

There are indigenous microorganisms in the carbon bearing subterraneous formations that naturally convert the carbon bearing materials into lower molecular weight hydrocarbons that are more easily recoverable than the nascent coal, such as methane, other gaseous or liquid hydrocarbons, or other valuable products. The microorganisms usually exist in the subterraneous formations as a consortium, meaning a mixture of multiple species of microorganisms that may depend on or interact with each other. One potentially practical way of using the residual carbon bearing material is by stimulating microorganisms in a subterraneous formation to more effectively metabolize the carbon bearing materials therein to produce compounds such as methane. Several methods have been developed for this purpose.

Certain methods involve the introduction of particular species or cultures of bacteria for treatment of carbon bearing materials. For example, U.S. Pat. No. 5,854,032 introduces a thermophilic aerobic culture ATCC 202096 to coal to convert the coal to humic acid.

U.S. Pat. No. 8,092,559 discloses a method for enhancing the microbial production of methane. The method includes steps of characterizing at least one environmental parameter for the in situ hydrocarbon-rich deposit, introducing an aqueous solution to the hydrocarbon-rich deposit located in the geologic formation, wherein the aqueous solution stimulates a microbial consortium to increase a production rate of methane from the in situ deposit, and collecting a gas mixture comprising the methane.

U.S. Pat. No. 8,176,978 discloses a process for in-situ production of methane, carbon dioxide, gaseous and liquid hydrocarbons, and other products from subterranean carbon bearing formations. The process comprises injecting fluid into a carbon bearing deposit via at least one injection well and removing injected fluid and product from the deposit through at least one production well. Fluid pressure within at least a portion of the deposit is controlled by use of the injected fluid such that the fluid pressure exceeds the fluid pressure that normally exists in that portion of the deposit.

WO 2011/142809 discloses a method of stimulating microbial consortia, such as microbial consortia in a geological formation, including, for example, methanogens and other bacteria, for producing methane and other hydrocarbon products, fuels or fuel precursors from coal or other carbonaceous materials wherein the consortia respond to electrical stimulation, either physically or chemically. Electrical energy is introduced into the carbonaceous formation to stimulate the growth of microbes or microbial consortia and a formed product is recovered from the formation.

U.S. 2010/0035309 discloses a process for biogenic production of a hydrogen-carbon-containing fluid from a hydrocarbon containing formation, comprising steps of providing an anaerobic microorganism consortium to the geologic formation containing one or more enzymes to activate a starting aromatic hydrocarbon by an addition of a chemical group to the starting aromatic hydrocarbon, converting the activated aromatic hydrocarbon into a hydrogen-carbon-containing fluid through one or more intermediate hydrocarbons and recovering the hydrogen-carbon-containing fluid from the formation.

U.S. Pat. No. 7,977,056 discloses a method of identifying a stimulant that increases the biogenic production of methane in a hydrocarbon-bearing formation. The method comprises obtaining a nucleic acid sequence from a microorganism derived from the formation, determining the presence of a gene product of the nucleic acid sequence, wherein the gene product is an enzyme in a pathway involved in conversion of hydrocarbon to methane, and identifying a substrate, reactant or co-factor of the enzyme that acts as a stimulant to increase methane production when provided to the microorganism in the formation as compared with methane production in the absence of the stimulant.

U.S. Pat. No. 7,832,475 describes a method for enhancement of methane production, comprising providing a hydrocarbon-bearing formation having at least two microbial populations, introducing at least one indiscriminate microbial population stimulation amendment to said formation, microbially consuming the stimulation amendment, microbially depleting the stimulation amendment, starving at least one of the at least two boosted microbial populations, selectively reducing said starved microbial population, selectively sustaining said at least one boosted microbial population, generating methane from said boosted microbial population, and collecting the methane.

Alternative methods are sought to potentially improve the yield, selectivity and/or rate of a process for converting carbon bearing materials to hydrocarbon products. The present disclosure describes alternative methods for converting the carbon bearing materials into one or more hydrocarbons, such as methane, for the purpose of potentially increasing the yield, selectivity and/or rate of the process, or to provide other advantages in the implementation of the process.

### SUMMARY

In a first aspect, the disclosure relates to a process involving a microorganism consortium for converting at least one component in a carbon-bearing material to a different product comprising at least one hydrocarbon. In the process a microorganism consortium is contacted with a composition that causes an increase or decrease of a relative population of at least one species of microorganism in said microorganism consortium relative to at least another species of microorganism in said microorganism consortium, to enhance a yield or selectivity or alter a rate of said process, as compared to an identical process carried out in the absence of said composition, wherein said composition is selected from a composition that directly or indirectly affects an intracellular pathway of said at least one species of microorganism and a composition that directly or indirectly affects an intercellular signaling pathway that involves said at least one species of microorganism.

In another aspect, the disclosure relates to a process involving a microorganism consortium for converting at least one component in a carbon-bearing material to a different product comprising at least one hydrocarbon. Environmental conditions of the microorganism consortium, such as oxygen conditions or other physical conditions such as temperature, pressure, and physiological states of said microorganism consortium, are modified (for example, restricted), in way(s) that cause an increase or decrease of a relative population of at least one species of microorganism in said microorganism consortium relative to at least another species of microorganism in said microorganism consortium, to enhance a yield or selectivity or alter a rate of said process, as compared to an identical process carried out in the absence of said composition.

In yet another aspect, a microorganism consortium of the disclosure is contacted with a physical signal that causes an increase or decrease of a relative population of at least one species of microorganism in said microorganism consortium relative to at least another species of microorganism in said microorganism consortium, to enhance a yield or selectivity or alter a rate of said process, as compared to an identical process carried out in the absence of said physical signal, wherein said physical signal is selected from sound waves and electromagnetic waves.

In another aspect, the disclosure relates to a process involving a microorganism consortium for converting at least one component in a carbon-bearing material to a different product comprising at least one hydrocarbon. The microorganism consortium is contacted with a composition comprising at least one biomolecule, such as a nucleic acid binding oligonucleotide for the targeting of nucleic acids and polypeptides, an antisense RNA, a nucleic acid analog that mimics antisense RNA, or a micro-RNA that causes an increase or decrease of a relative population of at least one species of microorganism in said microorganism consortium relative to at least another species of microorganism in said microorganism consortium, to enhance a yield or selectivity or alter a rate of said process, as compared to an identical process carried out in the absence of said composition.

### DETAILED DESCRIPTION OF THE PREFERRED EMBODIMENT(S)

For illustrative purposes, the principles of the present disclosure are described by referencing various exemplary embodiments. Although certain embodiments of the disclosure are specifically described herein, one of ordinary skill in the art will readily recognize that the same principles are equally applicable to, and can be employed in other systems and methods. Before explaining the disclosed embodiments of the present disclosure in detail, it is to be understood that the disclosure is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

As used herein, the term “carbon bearing material” includes any high carbon-content material that exists in a subterraneous formation. Examples of carbon bearing material include, but not limited to, oil shale, coal, coal seam, waste coal, coal derivatives, lignite, peat, oil formations, tar sands, hydrocarbon-contaminated soil, petroleum sludge, drill cuttings, and the like and may even include those conditions or even surroundings in addition to oil shale, coal, coal seam, waste coal, coal derivatives, lignite, peat, bitumen, oil formations, tar sands, hydrocarbon-contaminated soil, petroleum sludge, drill cuttings, and the like.

As used herein, “coal” refers to any of the series of carbonaceous fuels ranging from lignite to anthracite. The members of the series differ from each other in the relative amounts of moisture, volatile matter, and fixed carbon they contain. Coal is comprised mostly of carbon, hydrogen and entrained water, predominantly in the form of large molecules having numerous double carbon bonds. Low rank coal deposits are mostly comprised of coal and water. Energy can be derived from the combustion of carbonaceous molecules, such as coal, or carbonaceous molecules derived from the solubilization of coal molecules. The most useful coal includes coal containing the largest amounts of fixed carbon and the smallest amounts of moisture and volatile matter.

As used herein, the term “microorganism” includes bacteria, archaea and fungi. The microorganisms may be indigenous or exogenous to the carbon bearing materials. The microorganisms, by example, may include: Archaeoglobales, Thermotogales, Cytophaga group, Azospirillum group, Paracoccus subgroup, Sphingomonas group, Nitrosomonas group, Azoarcus group, Acidovorax subgroup, Oxalobacter group, Thiobacillus group, Xanthomonas group, Oceanospirillum group, Pseudomonas and relatives, Marinobacter hydrocarbonoclaticus group, Pseudoalteromonas group, Vibrio subgroup, Aeromonas group, Desulfovibrio group, Desulfuromonas group, Desulfobulbus assemblage, Campylobacter group, Acidimicrobium group, Frankia subgroup, Arthrobacter and relatives, Nocardiodes subgroup, Thermoanaerobacter and relatives, Bacillus megaterium group, Carnobacterium group, Clostridium and relatives, and archaea such as Methanobacteriales, Methanomicrobacteria and relatives, Methanopyrales, and Methanococcales.

More specific examples of microorganisms may include, for example, Aerobacter, Aeromonas, Alcaligenes, Bacillus, Bacteroides, Clostridium, Escherichia, Klebsiella, Leptospira, Micrococcus, Neisseria, Paracolobacterium, Proteus, Pseudomonas, Rhodopseudomonas, Sarcina, Serratia, Streptococcus and Streptomyces, Methanobacterium omelianskii, Mb. Formicium, Mb. Sohngenii, Methanosarcina barkeri, Ms. Methanica, Mc. Masei, Methanobacterium thermoautotrophicum, Methanobacterium bryantii, Methanobrevibacter smithii, Methanobrevibacter arboriphilus, Methanobrevibacter ruminantium, Methanospirillum hungatei, Methanococcus vannielli, Methanothrix soehngenii, Methanothrix sp., Methanosarcina mazei, Methanosarcina thermophila, Methanobacteriaceae, Methanosarcinaceae, Methanosaetaceae, Methanocorpusculaceae, Methaanomicrobiaceae, other archaea and a combination of these.

As used herein, the term “microorganism consortium” refers to microbes in a carbon bearing material, including a microorganism assemblage, containing two or more species or strains of microorganisms, and especially one in which each species or strain benefits from interaction with the other(s). The species or strains in the microorganism consortium may be indigenous to the carbon bearing material or exogenous to the carbon bearing material (introduced from external to the carbon bearing material).

As used herein, the term “bioconversion” or “conversion” refers to the conversion of carbon bearing materials into a product that may include methane and other useful gases and liquid components by a microorganism consortium in the carbon bearing material. The term “product” refers to a composition obtained from a carbon bearing material, such as coal, by bioconversion. The product includes, but not limit to, organic materials such as hydrocarbons, for example, methane, cetane, butane, and other small organic, as well as fatty acids, that are useful as fuels or in the production of fuels, as well as inorganic materials, such as gases, including hydrogen and carbon dioxide.

The conversion process may involve multiple reaction steps each of which may involve one or more microorganisms. In addition, the microorganisms directly involved in the conversion process may interact with other microorganisms involved in the conversion process or other microorganisms in the microorganism consortium that may be indirectly involved in the conversion process. Indirect involvement in the conversion process may entail competition with a microorganism directly involved in the conversion process for a nutrient or reactant, promotion or inhibition of a microorganism directly involved in the conversion process, and/or influencing the environment in which the microorganism consortium operates by changing a condition such as increasing or decreasing the presence of a toxin, food element, reactant, or changing a physical parameter, such as decreasing oxygen concentration or exposing the consortium to sound waves or electromagnetic current. In another aspect, the present invention may be used to influence signaling among microorganisms for example, by manipulation or alteration of quorum sensing mechanisms.

The present disclosure provides a method of converting at least some a carbon bearing material into a product that comprises at least one hydrocarbon. In one aspect, the method comprises the step of introducing a composition to the carbon bearing material for the purpose of interacting with a microorganism or a microorganism consortium therein.

In one aspect, the composition introduced to the carbon bearing material may cause an increase or decrease of a population of at least one species of microorganism. The increase or decrease of a population of at least one species of microorganism may be determined relative to a population of at least another species of microorganism in a microorganism consortium wherein both microorganisms are present or may be determined on an absolute basis by comparison of the populations of that microorganism prior to and after introduction of the composition to the carbon bearing material.

The adjustment of the population of at least one species of microorganism may be used to, for example, enhance yield, selectivity, or alter a reaction rate of the process for conversion of a carbon bearing material to a hydrocarbon product. This may be determined in comparison with an identical process carried out in the same carbon bearing formation without the introduction of the composition thereto.

The adjustment of a population of at least one species may also be used to enhance a population of a particular microorganism that is involved in a rate-limiting step of the conversion process. This microorganism may be enhanced by increasing its population, by decreasing the population of a microorganism that competes for its nutrients and/or competes for one or more reactants used by that microorganism in its participation in the conversion process. By decreasing competition with the microorganism, the population of the microorganism may increase and/or the same population of microorganism may be able to provide an increased yield to due improved access to nutrients and/or needed reactants.

In another embodiment, a composition can be introduced for the purpose of increasing a nutrient, decreasing a concentration of a toxin, promoting a favorable microorganism and/or inhibiting a competing microorganism in the consortium. Thus, in one aspect a particular nutrient component may be identified as suitable for a particular microorganism in the consortium and a supply of that nutrient may be increased by the composition. For example, the composition may inhibit a competing microorganism that relies on the same nutrient. Alternatively, the composition may promote growth of a microorganism that supplies the nutrient.

In another aspect, a particular toxin or antibiotic may be identified which is harmful to, or inhibits the activity of a particular microorganism in the consortium and the introduced composition may contain a component directed to reducing the concentration of that toxin or antibiotic in the carbon bearing material. For example, a material that binds to or reacts with the toxin or antibiotic could be useful for this purpose. Also, materials that absorb or neutralize the toxin would be useful.

In another aspect, the composition that is introduced may be used to promote a population and/or activity of a favorable microorganism. In one embodiment, component could be introduced which enhances the population and/or activity of a microorganism that consumes an undesirable toxin. In another aspect, the composition may be used to promote a population and/or activity of a microorganism that converts an undesirable by-product of the process into one or both of a desirable end product and a product useful as a reactant in the carbon bearing material conversion process. In this manner, undesirable by-products can be converted to desirable end products or can be cycled back into the carbon bearing material conversion process and converted to desirable end products therein.

In another aspect, the composition that is introduced may be used to inhibit a population or activity of an unfavorable microorganism. Such an unfavorable microorganism may be a microorganism that promotes the generation of an undesirable by product. Such an undesirable microorganism may be one which inhibits the population and/or activity of a desirable microorganism or a microorganism that generates an undesirable toxin such as hydrogen sulfide.

The conversion of carbon bearing material to the product may be carried out in situ, i.e. in a geologic or subterraneous formation where the carbon bearing material is naturally present. The conversion also may be carried out ex situ, i.e. in a location other than where the carbon bearing material is naturally present. Ex situ conversion may be carried out in places such as a bioreactor, an ex situ reactor, a pit, an aboveground structure, and the like. For example, the carbon bearing material may first be removed from the location where it is naturally present and then subjected to the method of the present disclosure. As a non-limiting example, a bioreactor may refer to any device or system that supports a biologically active environment.

In one aspect of the method, the composition may be introduced to the carbon bearing material by any suitable method. In one embodiment, the composition may be introduced as a fluid to the carbon bearing material. Fluids may be introduced by injection into the carbon bearing material. In another embodiment, the composition may be in solid form and may be located proximate to the carbon bearing material, where fluid may dissolve and/or distribute the composition to the carbon bearing material. In yet another embodiment, the composition may be delivered as an aerosol and may be introduced by blowing the composition into contact with the carbon bearing material. Suitable methods that may be employed to introduce the compositions of the invention to the carbon bearing material such as those described, for example, in U.S. 2010/000732, U.S. 2010/032157, U.S. 2012/043084, and U.S. 2012/0199492, the disclosures of which are hereby incorporated by reference herein.

Flow speed can be regulated to affect concentration of introduced compositions or existing signaling molecules, or to modify physical signals delivered to a microbial consortium or environmental conditions of a microbial consortium. For example, fluid flow speed (or movement) can be modified, changing the concentration of signaling molecules directly or indirectly. In particular, in a set-up comprising a fill reservoir to deliver composition(s) or physical signal(s) to the coal, and a recovery reservoir (at the same site or at different sites) to recover the product. Compositions, including nutrients or other compositions, can be delivered at desired concentrations, and pumping processes, fluid movement and resident fluid(s) dilutions can be modified to further modify composition or signaling molecule concentrations in-situ. Fluids and fluid dilutions can be designed ex-situ. Once desired concentrations are reached, product can be generated and recovered.

The microorganism(s) and/or microorganism consortium in the carbon bearing material may be entirely indigenous, wherein all species of microorganisms in the carbon bearing material are naturally present therein, or, in some embodiments, the microorganism(s) and/or consortium in the carbon bearing material may include at least one exogenous species, or at least one species with its population supplemented with exogenous microorganisms.

The microorganism(s) and/or microorganism consortium in the carbon bearing material are responsible for aspects of the conversion of the carbon bearing material to a hydrocarbon product, which typically occurs via a thermochemical process that is influenced by the activity of various microorganisms. A plurality of different species in the microorganism consortium may play a role and/or make a contribution to the conversion process. Also, each individual species may play a role and/or make a contribution to the conversion process. Further, each individual species may influence the interaction among different species or may influence one or more other species in a way that may alter the population and/or effectiveness of that species in the microorganism consortium.

For example, in a particular microorganism consortium, one or more species of microorganism may be capable of enhancing the yield or selectivity of the conversion process. This enhancement may be brought about in one or more of several different ways. For example, in one embodiment a particular microorganism is associated with a rate-limiting step of the conversion process and an increase in the population of that microorganism may increase yield from the rate-limiting step thereby increasing the overall yield, rate and/or selectivity of the conversion process. Alternatively, the reaction rate of a process step that produces an undesirable by-product can be reduced by the method of the invention.

In another embodiment, it may be desirable to promote growth of a microorganism that produces a desirable extracellular signaling, such as a signal that enhances growth of a microorganism that participates in a rate-limiting step of the conversion reaction. In this manner, it may be possible to indirectly influence the population of a desirable microorganism.

In another aspect, at least one species of microorganism may have an inhibitory effect on the yield or selectivity of the converting process. When the relative population of useful species of microorganism in the consortium is increased, the overall yield or selectivity of the conversion process may be enhanced. On the other hand, decreasing the relative population of inhibitory species of microorganism may also enhance the overall yield, alter a reaction rate, or selectivity of the conversion process. One study of microorganisms and their roles on converting methane production from a carbon source may be found in WO WO/2011/159924, which is hereby incorporated by reference herein its entirety.

Binding proteins are known that modify cell division in stressful environments, such as low oxygen environments. For example, in low oxygen environments, the protein HIF-1alpha binds to a protein that loads a DNA replication complex onto DNA strands, preventing the complex from being activated, thus stopping cells from dividing (M. E. Hubbi, et al., “A Nontranscriptional Role for HIF-1 as a Direct Inhibitor of DNA Replication,” Science Signaling, 2013; 6 (262)).

In one aspect of the present disclosure, the composition comprises at least one protein, such as a binding protein, that is capable of causing an increase or decrease of relative population of at least one species of microorganism in the microorganism consortium relative to at least another species of microorganism in the microorganism consortium. In one aspect of the present disclosure, the protein is an enzyme. The enzyme may be selected from enzymes that create a condition that favors or disfavors at least one species in the microorganism consortium, enzymes that affects an intracellular pathway of at least one species of microorganism, and enzymes that affect an intercellular signaling pathway that involves at least one species of microorganism.

The enzymes that are suitable for the present invention may include Acetyl xylan esterase, Alcohol oxidases, Allophanate hydrolase, Alpha amylase, Alpha mannosidase, Alpha-L-arabinofuranosidase, Alpha-L-rhamnosidases, Ammoniamonooxygenase, Amylases, Amylo-alpha-1,6-lucosidase, Arylesterase, Bacterial alpha-L-rhamnosidase, Bacterial pullanases, Beta-galactosidase, Beta-glucosidase, Carboxylases, Carboxylesterase, Carboxymuconolactone decarboxylase, Catalases, Catechol dioxygenase, Cellulases, Chitobiase/beta-hexo-aminidase, CO dehydrogenase, CoA ligase, Dexarboxylases, Dienelactone hydrolase, Dioxygenases, Dismutases, Dopa 4,5-dioxygenase, Esterases, Family 4 glycosylhydrolases, Glucanaeses, Glucodextranases, Glucosidases, Glutathione S-transferase, Glycosyl hydrolases, Hyaluronidases, Hydratases/decarboxylases, Hydrogenases, Hydrolases, Isoamylases, Laccases, Levansucrases/Invertases, Mandelate racemases, Mannosyl oligosaccharide glucosidases, Melibiases, Methanomicrobialesopterin S-methyltransferases, Methenyl tetrahydro-methanopterin cyclohydrolases, Methyl-coenzyme M reductase, Methylmuconolactone methyl-isomerase, Monooxygenases, Muconolactone delta-isomerase, Nitrogenases, O-methyltransferases, Oxidases, Oxidoreductases, Oxygenases, Pectinesterases, Periplasmic pectate lyase, Peroxidases, Phenol hydroxylase, Phenol oxidases, Phenolic acid decarboxylase, Phytanoyl-CoA dioxygenase, Polysaccharide deacetylase, Pullanases, Reductases, Tetrahydromethan-opterin S-methyltransferase, Thermotoga glucanotransferase and Tryptophan 2,3-dioxygenase.

In some exemplary embodiments, the enzyme selected for use in the composition can create a condition that favors or disfavors at least one species in the microorganism consortium. The enzyme may achieve this purpose by transforming a component in the carbon bearing material into either a substance that promotes growth of at least one species of microorganism to enhance the yield, rate or selectivity of the conversion process, or a substance that inhibits growth of at least one species inhibitory to the yield, rate and/or selectivity of the conversion process.

In some other exemplary embodiments, the enzyme may destroy a component in the carbon bearing material that inhibits growth of at least one species of microorganism that promotes the yield, rate and/or selectivity of the conversion process, or a component that promotes growth of at least one species inhibitory to the yield, rate and/or selectivity of the process. In this manner, the enzyme may be used to indirectly influence the relative population of one or more species of microorganisms in the microorganism consortium.

In other embodiments, the enzyme in the composition may be used to interfere with extracellular signaling among the species in the microorganism consortium. The plurality of species in a microorganism consortium is like a community where the species communicate with, and to some extent, interact with and depend upon, each other. Using an enzyme to disrupt extracellular signaling among certain microorganism may be used to alter the balance in the community to thereby manipulate the microorganism consortium to increase the yield, rate and/or selectivity of the conversion process.

Bacteria are known to have ways of communicating with each other via signaling systems. For example, one such signaling system that inhibits formation of biofilm causes bacteria to produce a flagellum that gives the bacteria the ability to swim away (Jindong Zan, et al., “A complex LuxR-LuxI type quorum sensing network in a roseobacterial marine sponge symbiant activates flagellar motility and inhibits biofilm formation,” Molecular Microbiology, vol. 85, page 916, 2012).

In an exemplary embodiment, the targeted extracellular signaling is quorum sensing, through which a microorganism detects and responds to chemical molecules called autoinducers present in the environment in a dose dependent fashion. Autoinducers can be produced by microorganisms of the same species, or of different species. When the concentration of an autoinducer reaches a critical threshold, a microorganism detects the autoinducer and responds to this signal by altering its gene expression. Quorum sensing allows microorganisms in a consortium to behave as a collective community similar to a multicellular entity.

Quorum sensing is different among different groups of microorganisms in the microorganism consortium. For example, gram-negative bacteria may use a LuxIR system, which has acyl homoserine lactones (AHL) as the autoinducer. AHL has a common homoserine lactone moiety but variable acyl side chains. Gram-negative bacteria use the LuxI protein, or a homolog of this protein, to synthesize AHL, while using LuxR (or a homologue of LuxR) as a regulator that binds to the autoinducer and modulates gene expression within the bacteria. This LuxIR system demonstrates great specificity, as the AHL produced by one species can rarely, if ever, interact with the LuxR regulator of another species.

Gram-positive bacteria use an oligopeptide system, which uses peptides as autoinducer. The peptides are produced in cytoplasm as precursor peptides and then cleaved, modified and exported into the environment. The autoinducers are detected by a two-component complex which has an external portion of a membrane-bound sensor kinase protein that detects the autoinducer, and then phosphorylates/activates a response regulator that modulates gene expression within the bacteria. The peptide autoinducer also appears to be specific to the species that produces it.

A third major quorum sensing system is found in wide variety of bacteria, including both gram-negative and gram positive species, the LuxS system, which uses the autoinducer AI-2. AI-2 is detected by a two-component system LuxP/LuxQ (regulator), and the resulting phosphorylation cascade leads to modulation of gene expression.

Bacterial growth is often dependent on the quorum system. For example, some bacteria grow well in a community but cannot be easily cultured from a single bacterial cell. It appears that the bacterial growth of certain bacteria is arrested when these bacteria do not detect certain autoinducers in the environment via the quorum sensing system.

In one embodiment, the present disclosure uses an enzyme to disrupt the quorum sensing system of at least one bacterial species in order to specifically inhibit the growth of the at least one bacterial species. An enzyme may be used to target various aspects of the quorum sensing system, especially the extracellular portion. In an exemplary embodiment, an enzyme is used to specifically degrade an autoinducer of a particular species of bacteria which is linked to the growth of that species of bacteria. The growth of that species will thus be inhibited since it will not detect a sufficient amount of the required autoinducer in the environment.

In another exemplary embodiment, an enzyme may be used to specifically degrade the regulator of a bacteria species. Because the species relies on the regulator to detect autoinducers, bacteria with degraded regulator will not be able to detect the autoinducer in the environment. Thus the growth of that species of bacteria species may also be inhibited in this manner. In certain embodiments, an enzyme may be able to degrade multiple autoinducers and/or multiple regulators that share a common moiety, and thus the growth of multiple species of bacteria may be inhibited by a single enzyme. Alternatively, multiple enzymes may be used to degrade multiple autoinducers and/or multiple regulators in a microorganism consortium to thereby inhibit growth of multiple species of bacteria.

Hazan, R., et al., “Homeostatic Interplay between Bacterial Cell-Cell Signaling and Iron in Virulence,” (2010), PLoS Pathog. 6(3): e1000810, dio:10.1371/journal.ppat. 100810 describes a methodology for identifying compositions that participate in quorum sensing signaling pathways. Also, Kaper, J. B. and Sperandio, V., “Bacterial Cell-to-Cell Signaling in the Gastrointestinal Tract,” Infection and Immunity, June 2005, pp. 3197-3209 describes characterization of quorum sensing and compositions participating therein. This article demonstrates that the quorum sensing system, autoinducers and regulated phenotypes for particular bacterial species can be identified using existing methods. The disclosures of these references are hereby incorporated by reference in their entirety.

Increases in relative population of relevant members of a microorganism consortium may also be achieved by activating autoinducers. For example, the element borate has been found to cause an AI-2 precursor to generate active AI-2, a ‘universal’ signal for inter-species communication (Chen X., et al., Nature Jan. 31, 2002; 415(6871): 545-9, incorporated herein by reference in its entirety).

Bacteria are known to cooperate among individuals against competing populations. In Science, 7 Sep. 2012: Vol. 337 no. 6099 pp. 1228-1231, Cordero, et al. showed that broad range antibiotics were produced by a few genotypes in a population whereas other genotypes were resistant, suggesting cooperation between conspecifics. Antibiotics produced in this way may thus mediate competition between populations rather than solely increase the success of individuals (“Ecological Populations of Bacteria Act as Socially Cohesive Units of Antibiotics Production and Resistance”). The disclosure of this reference is hereby incorporated by reference in its entirety.

In another aspect of the present disclosure, the composition may include at least one antibiotic that is capable of causing a decrease in the relative population of at least one species of microorganism in the microorganism consortium relative to at least another species of microorganism in the microorganism consortium. The populations of certain classes of microorganisms may be reduced by introducing antibiotics into the microorganism consortium.

Suitable antibiotics for the present invention include ampicillin, chloramphenicol, erythromycin, fosfomycin, gentamicin, kanamycin, neomycin, penicillin, rifampicin, streptomycin , tetracycline and vancomycin.

In one embodiment of the present invention, the population is exposed to physical signals, such as sound waves or electromagnetic current. Experimental evidence is available that indicates that microbes can generate and respond to these physical signals (Trends Microbiol., 2011 March: 19(3); 105-113 “When Microbial Conversations get Physical”, incorporated herein by reference herein in its entirety).

In one embodiment, the composition may include at least one biomolecule, such as a nucleic acid-binding oligonucleotide for the targeting of nucleic acids and polypeptides, an antisense RNA, a nucleic acid analog that mimics antisense RNA, or a micro-RNA that is capable of causing an increase or decrease of relative population of at least one species of microorganism in the microorganism consortium relative to at least another species of microorganism in the microorganism consortium. Biomolecules may be used to inhibit the growth of one or more species of microorganisms or to promote the growth of one or more species of microorganisms. Due to the high specificity of some biomolecules, such as nucleic acid binding oligonucleotides, the growth inhibition may be only for a single species or a group of species, for example those species that have a nucleic acid that shares the same sequence domain that binds to the nucleic acid binding oligonucleotide.

In one exemplary embodiment, the present disclosure uses a nucleic acid binding oligonucleotide to target the nucleic acid of a component in a metabolic pathway of a species of microorganism. The disruption of the metabolic pathway in the species will inhibit the growth of the microorganism. In yet another exemplary embodiment, the present disclosure uses a nucleic acid binding oligonucleotide to target a component in a signaling pathway of a species of microorganism. The disruption of the signaling pathway in the species will inhibit the growth of the microorganism. There are many metabolic pathways and signaling pathways that may be targeted for disrupting the growth of microorganisms. The nucleic acid binding oligonucleotides may target proteins or nucleic acids in one or more of these metabolic or signaling pathways.

Nucleic acid binding oligonucleotides may also be used to disrupt the extracellular signaling, such as quorum sensing. For example, nucleic acid binding oligonucleotides may be used to target the nucleic acid of a protein involved in the synthesis of an autoinducer, in order to reduce or prevent synthesis of the autoinducer. Alternatively, nucleic acid binding oligonucleotides may be used to target the nucleic acid of a regulator that detects the autoinducer. When there is no autoinducer in the environment or the microorganism has no regulator to detect the autoinducer, the quorum sensing is disrupted. Therefore, the growth of a species for which growth is dependent on quorum sensing, may be inhibited in this manner.

In one embodiment, the FRET system may be used to identify nucleic acid molecules that can hybridize with RNAs within a bacteria species, such as ribosomal RNA, especially its A-site. These nucleic acid molecules can be used as antibiotics to specifically inhibit the growth a class or species of bacteria.

Nucleic acid binding oligonucleotides may be used in many other ways to inhibit the growth of a species of microorganism. For example, the nucleic acid of a structural protein may be targeted by one or more nucleic acid binding oligonucleotides. The lack of the structural protein may cause inhibition of the growth of the microorganism. In another example, the nucleic acid of a protein that is involved in microorganism reproduction may be targeted, to thereby inhibit microorganism reproduction.

In some embodiments of the present disclosure, the nucleic acid binding oligonucleotides may be modified to enhance the uptake of the nucleic acid binding oligonucleotides by the cells of a microorganism. One way of modifying the nucleic acid binding oligonucleotides is by covalent linking to a delivery agent. For example, nucleic acid binding oligonucleotides may be conjugated with a peptide transduction domain (PTD) that facilitates the uptake of the nucleic acid binding oligonucleotides (see Meade et al., “Enhancing the cellular Uptake of siRNA Duplexes Following Noncovalent Packaging with Protein Transduction Domain Peptides,” Adv. Drug Deliv. Rev., March 2008 1; 60(4-5): 530-536). Other suitable delivery agents include the Minis Transit TKO lipophilic agent; lipofectin; lipofectamine; cellfectin; and polycations (e.g., polylysine).

Liposomes can also be used to aid the delivery of nucleic acid binding oligonucleotides to the cells of microorganisms. Liposomes suitable for use in the disclosure are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, as described in U.S. Pat. Nos. 4,235,871, 4,501,728, 4,837,028, and 5,019, 369, which are incorporated herein by reference in their entirety.

In yet other embodiments, nucleic acid binding oligonucleotides may be expressed from a plasmid that is introduced inside the cells of a microorganism. Any plasmid vector that is capable of expressing nucleic acid binding oligonucleotides in a microorganism cell may be used in the present disclosure.

In yet other embodiments, a viral expression vector may be used to deliver a nucleic acid binding oligonucleotides into microorganism cells. Any viral vector capable of accepting the coding sequences for the nucleic acid binding oligonucleotides to be expressed in the microorganism can be used. Bacteriophages are a suitable example of a viral expression vector. After the viral vector enters the microorganism cells, nucleic acid binding oligonucleotides may be produced from the vector.

The method of the present invention may include steps of selecting one or more microorganisms and identifying one or more compositions useful for influencing the population of the microorganism.

In one aspect, the method of the present invention selects one or more microorganisms for which it is desirable to influence the population. These microorganisms may be selected on the basis of a variety of different criteria. Thus, microorganisms may be selected based on their direct participation in the process of conversion of carbon bearing materials to hydrocarbons or based on indirect participation in the process. For example, microorganisms that compete with desirable microorganisms for nutrients and/or raw materials may be selected for population adjustment. Microorganisms that produce toxins or antibiotics or otherwise adversely influence the environment for the conversion reaction may be selected. Microorganisms that produce desirable extra-cellular signaling may also be selected. Also, microorganisms may be selected based on the amounts or types of enzymes or proteins that they produce or based on the waste materials that they generate.

Once a particular microorganism or group of microorganisms is selected for population adjustment, the present method may then determine whether the population of a particular microorganism is to be increased or decreased based on one or more of the criteria discussed above. Once this is determined, various strategies in accordance with the present method may be employed to achieve this goal.

After identification of the microorganism, the present invention may identify an intracellular pathway of the species of microorganism extracellular signaling pathway of the microorganism that can be manipulated to achieve the desired goal. Once such a pathway is identified, a necessary component or aspect of that pathway can be identified for inhibition. For example, a particular autoinducer or regulator that participates in detection of an autoinducer can be targeted to influence an extra-cellular quorum sensing pathway. Other signaling agents may also be identified and targeted. Alternatively, a component of an intercellular pathway can be identified and targeted or a receptor uses for a pathway can be targeted or blocked.

Alternatively, a target for an antisense RNA in said microorganism can be identified. Once the target is identified, a suitable antisense RNA can be selected and employed to influence the population of the microorganism. Suitable targets for antisense RNA can be, for example, components found in the mitochondria or that participate in intracellular or intercellular signaling. Also, components of the cell that participate in, for example, enzyme production can be targeted for inhibition.

A further alternative is to select an antibiotic that targets the selected microorganism. Preferably, a selective antibiotic is selected for this purpose so as to specifically target a particular microorganism.

Patent application publication number WO2008/133709 entitled, “Targeted Split Biomolecular Conjugates for the Treatment of Diseases, Malignancies and Disorders, and Methods of their Production” discloses types of compositions that are useful for the methods of the present invention. The compositions are split-biomolecular conjugates for the directed targeting of nucleic acids and polypeptides. The split biomolecular conjugates comprise split effector protein fragments conjugated to a probe. Interaction of both probes with a target nucleic acid or target polypeptide, such as a pathogenic nucleic acid sequence or pathogenic protein, brings split-effector fragments together to facilitate the reassembly of the effector molecule. Depending on the effector molecule, the protein complementation results in a cellular effect. In the method of the present invention, such compositions can be employed as described herein.

Once the composition(s) to be used to influence microorganism population are identified, the composition(s) are formulated into a suitable composition for delivery to the carbon bearing material. Suitable compositions are described above.

Another consideration for selection of suitable components for use in the present invention is their potential influence on other species of microorganisms present in the microorganism consortium. Thus, in some aspects of the present disclosure, additional testing or analysis may be conducted to determine the effects of a proposed component on other species of microorganisms present in the microorganism consortium. For this purpose, a simulation of the reaction can be conducted using a computer or other suitable means, or a small-scale conversion reaction can be set up and tested for the results of the introduction of particular components to the conversion reaction.

In one aspect of the present disclosure, the composition introduced to the carbon bearing material comprises at least one nutrient that is capable of causing an increase or decrease of the population of at least one species of microorganism in the microorganism consortium relative to at least one other species of microorganism in the microorganism consortium.

There are different nutrient requirements for different species of microorganisms in the microorganism consortium. As a result, particular nutrients can be selected to manipulate the microorganism consortium in a particular way based on knowledge of the particular microorganisms and their nutrient requirements. In this manner, specific nutrients can be employed to influence the relative populations of at least some of the species in the microorganism consortium for the purpose of, for example, enhancing the yield, selectivity or altering a rate of a reaction.

The nutrients may be substances upon which one or more species of microorganism is dependent or the nutrients may substances that can or will be converted to a substance upon which one or more species of microorganism is dependent. Conversely, the nutrients may be themselves be substances that hinder a species of microorganism that is inhibitory to the yield, selectivity or rate of the conversion process or the nutrients may be converted to a substance that hinders a species of microorganism that is inhibitory to the yield, selectivity or rate of the conversion process.

Suitable nutrients for the present invention include ammonium, ascorbic acid, biotin, calcium, calcium pantothenate, chlorine, cobalt, copper, folic acid, iron, K₂HPO₄, KNO₃, magnesium, manganese, molybdenum, Na₂HPO₄, NaNO₃, NH₄Cl, NH₄NO₃, nickel, nicotinic acid, p-aminobenzoic acid, phosphorus, potassium, pyridoxine HCL, riboflavin, selenium, sodium, thiamine, thioctic acid, tungsten, vitamin B12, vitamins and zinc.

Thus, in one aspect of the present method, the composition may be introduced in addition to, or in combination with, one or more species of microorganisms in order to influence the conversion process. Additional species of microorganisms may be provided for a variety of different purposes. For example, a particular microorganism that is involved in a rate-limiting step of the conversion process may be supplemented to increase the reaction rate or yield of that rate-limiting step. In another embodiment, a particular microorganism can be introduced for the purpose of increasing a nutrient, decreasing a concentration of a toxin, and/or inhibiting a competing microorganism for different microorganism in the consortium that participates in the conversion process. One or more species of microorganisms may be introduced to accomplish two or more of these purposes.

In some embodiments, studies or computer simulations of the conversion process and/or the environment for the conversion process may be employed to select a particular composition for use in the present disclosure. For example, the method described in U.S. 2010/0081184, the disclosure of which is hereby incorporated by reference, may be employed for this purpose.

In some embodiments of the present disclosure, the carbon bearing material may be pretreated to increase permeability of the carbon bearing material, thus increasing the susceptibility of the large carbonaceous molecules in the carbon bearing material to be converted by a microorganism consortium. Physical (e.g., fracture and the like) and chemical approaches (e.g., treating with surfactants, acids, bases, oxidants, such as but not limited to acetic acid, sodium hydroxide, percarbonate, peroxide and the like) can be applied to enhance an availability of organic matter in carbon bearing material such as coal and oil shale. These methods may be used to break down coal, oil shale, lignite, coal derivatives and like structures to release more organic matter, or perhaps even to make them more vulnerable to degradation into smaller organic compounds. Some suitable pretreatment methods are described in U.S. 2010/0139913, WO 2010/1071533 and U.S. 2010/0262987, the disclosures of which are hereby incorporated by reference herein.

In addition, the present invention may be used in conjunction with other methods for altering the bioconversion of carbon bearing materials, such as, for example, the electro-stimulation method described in WO 2011/142809, the disclosure of which is hereby incorporated by reference herein.

It is to be understood, however, that even though numerous characteristics and advantages of the present disclosure have been set forth in the foregoing description, together with details of the structure and function of the disclosure, the disclosure is illustrative only, and changes may be made in detail, especially in matters of shape, size and arrangement of parts within the principles of the disclosure to the full extent indicated by the broad general meanings of the terms in which the appended claims are expressed.

1. A process involving a microorganism consortium for converting at least one component in a carbon-bearing material to a different product comprising at least one hydrocarbon, said process comprising the step of:
contacting said microorganism consortium with a composition that causes an increase or decrease of a relative population of at least one species of microorganism in said microorganism consortium relative to at least another species of microorganism in said microorganism consortium, to enhance a yield or selectivity or alter a rate of said process, as compared to an identical process carried out in the absence of said composition,
wherein said composition is selected from a composition that directly or indirectly affects an intracellular pathway of said at least one species of microorganism and a composition that affects an intercellular signaling pathway that involves said at least one species of microorganism.

2. The process of claim 1, wherein said intracellular pathway is a metabolic pathway.

3. The process of claim 1, wherein said intracellular pathway is a signaling pathway.

4. The process of claim 1, wherein said intercellular signaling pathway is a Quorum sensing pathway.

5. The process of claim 1, wherein said composition affects an autoinducer of said at least one species of microorganism.

6. The process of claim 5, wherein said composition affects a regulator of said at least one species of microorganism and said regulator is required by said microorganism to detect an autoinducer.

7. The process of claim 1, wherein said composition comprises at least one enzyme that affects an intracellular pathway of said at least one species of microorganism and a composition that affects an intercellular signaling pathway that involves said at least one species of microorganism.

8. The process of claim 1, wherein said carbon-bearing material is located in a subterranean formation.

9. The process of claim 1, wherein said carbon-bearing material is located in an ex-situ formation.

10. The process of claim 1, wherein said carbon-bearing material comprises coal.

11. The process of claim 1, wherein said product comprises methane.

12. The process of claim 1, wherein said composition comprises at least one antibiotic to decrease the relative population of said at least one species of microorganism in said microorganism consortium relative to said at least another species of microorganism in said microorganism consortium.

13. The process of claim 1, wherein said composition is in a liquid form.

14. The process of claim 1, wherein said composition is in a solid form.

15. The process of claim 1, wherein said composition is in an aerosol form.

16. The process of claim 1, wherein said composition further comprises at least one nutrient.

17. The process of claim 1, further comprising the steps of:
(a) identifying a microorganism for population adjustment,
(b) identifying an intracellular pathway or extracellular signaling pathway of said microorganism identified in step (a), and
(c) identifying a component that influences the intracellular pathway or extracellular signaling pathway identified in step (b).

18. The process of claim 17, wherein in step (c), a target that participated in the intracellular pathway or extracellular signaling pathway is first identified and then a component is identified that is capable of inhibiting the target.

19-37. (canceled)

38. The process of claim 1, wherein the composition comprises at least one biomolecule.

39. The process of claim 38, wherein the at least one biomolecule is selected from a nucleic acid binding oligonucleotide, an antisense RNA, a nucleic acid analog that mimics antisense RNA and a micro-RNA.
